(19) United States
(12) Patent Application Publication
(10) Pub. No.: US 2026/0263050 A1
(43) Pub. Date: Sep. 10, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Kaito TAKASHIMA, Kanagawa (JP)

(73) Assignee: FUJIFILM Corporation, Tokyo (JP)

(21) Appl. No.: 19/558,358

(22) Filed: Mar. 5, 2026

(30) Foreign Application Priority Data
Mar. 6, 2025 (JP) 2025-035648

(51) Int. Cl. A61B 8/00; A61B 8/06; G06T 7/00; G06T 7/246

(52) U.S. Cl. CPC A61B 8/54; A61B 8/06; A61B 8/461; A61B 8/488; G06T 7/0012; G06T 7/248; G06T 2207/10132; G06T 2207/30048; G06T 2207/30104

(57) ABSTRACT

Provided are an ultrasound diagnostic apparatus and a control method of an ultrasound diagnostic apparatus that can easily and accurately select an ultrasound image of a frame representing a systolic mid-phase.

The ultrasound diagnostic apparatus includes a feature recognition unit that recognizes an anatomical feature of a heart in each of a plurality of frames, a temporal change acquisition unit that acquires a temporal change of the anatomical feature, a blood flow maximum phase specifying unit that specifies a blood flow maximum phase in a cardiac cycle by using time-varying information of an aortic blood flow volume, a systolic mid-phase specifying unit that specifies a systolic mid-phase in the temporal change by associating the blood flow maximum phase with the temporal change, a candidate frame extraction unit that extracts a measurement candidate frame representing the systolic mid-phase, and a display controller that displays the measurement candidate frame in a highlighted manner on a monitor.

### CROSS-REFERENCE TO RELATED APPLICATIONS

The present application claims priority under 35 U.S.C. § 119 to Japanese Patent Application No. 2025-035648, filed on Mar. 6, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method of the ultrasound diagnostic apparatus.

### 2. Description of the Related Art

In the related art, a so-called cardiac output is calculated by capturing an ultrasound image representing a tomographic plane of a heart of a subject using a so-called ultrasound diagnostic apparatus and analyzing the captured ultrasound image. The cardiac output is usually calculated by performing calculation steps of (1) measuring a diameter of a left ventricular outflow tract in an ultrasound image representing a so-called parasternal left ventricular long-axis cross section of a systolic mid-phase of the heart, and calculating a cross-sectional area of the left ventricular outflow tract, (2) calculating a velocity-time integral value of blood flow in the left ventricular outflow tract for a so-called apical five-chamber cross section by a so-called pulse Doppler method, (3) calculating a so-called stroke volume as a product of the cross-sectional area of the left ventricular outflow tract and the velocity-time integral value of the blood flow in the left ventricular outflow tract, and (4) calculating the cardiac output as a product of the stroke volume and a heart rate.

In a case in which the cardiac output is calculated in this way, for example, a problem may occur in which a criterion for selecting the ultrasound image of the frame representing the systolic mid-phase of the heart varies for each user of the ultrasound diagnostic apparatus, such as a doctor, or a large amount of time is required to select the ultrasound image of the frame representing the systolic mid-phase of the heart. It is generally known that the systolic mid-phase of the heart tends to be located in the vicinity of a phase in which the diameter of the left ventricular outflow tract is maximal. JP2023-054549A discloses a technique of automatically selecting the ultrasound image of the frame in which the diameter of the left ventricular outflow tract is maximal, and for example, by using this technique, a frame in the vicinity of the phase in which the diameter of the left ventricular outflow tract is maximal can be selected.

### SUMMARY OF THE INVENTION

Here, the diameter of the left ventricular outflow tract is not necessarily maximal at the systolic mid-phase, and a diameter of the same degree as the diameter of the left ventricular outflow tract at the systolic mid-phase may be measured in the ultrasound images of the plurality of frames. Therefore, even in a case in which the technique disclosed in JP2023-054549A is used, the ultrasound image of the frame representing the systolic mid-phase may not be selected, and the ultrasound image of the frame corresponding to a different phase may be selected each time the ultrasound image is selected.

The present invention has been made to solve such a problem in the related art, and an object of the present invention is to provide an ultrasound diagnostic apparatus and a control method of an ultrasound diagnostic apparatus that can easily and accurately select an ultrasound image of a frame representing a systolic mid-phase.

The above object can be achieved with the following configurations.

[1] An ultrasound diagnostic apparatus that guides a systolic mid-phase frame from ultrasound images of a plurality of frames in which a heart of a subject is imaged, the ultrasound diagnostic apparatus including: a feature recognition unit that recognizes an anatomical feature of the heart in each of the plurality of frames; a temporal change acquisition unit that acquires a temporal change of the anatomical feature recognized by the feature recognition unit; a blood flow maximum phase specifying unit that specifies a blood flow maximum phase in a cardiac cycle using time-varying information of a blood flow volume in an aorta; a systolic mid-phase specifying unit that specifies the systolic mid-phase in the temporal change by associating the blood flow maximum phase specified by the blood flow maximum phase specifying unit with the temporal change acquired by the temporal change acquisition unit; a candidate frame extraction unit that extracts a measurement candidate frame representing the systolic mid-phase specified by the systolic mid-phase specifying unit from the plurality of frames; a monitor; and a display controller that displays the measurement candidate frame extracted by the candidate frame extraction unit in a highlighted manner on the monitor.

[2] The ultrasound diagnostic apparatus according to [1], in which the temporal change acquisition unit specifies a diastolic-end phase and an systolic-end phase in the temporal change, and the systolic mid-phase specifying unit specifies a phase at which forward flow starts increasing and a phase at which the forward flow ends decreasing as the diastolic-end phase and the systolic-end phase, respectively, based on the time-varying information of the blood flow volume in the aorta, and calculates a time ratio of an elapsed time from the diastolic-end phase to the blood flow maximum phase specified by the blood flow maximum phase specifying unit to an elapsed time from the diastolic-end phase to the systolic-end phase, and specifies the systolic mid-phase based on the diastolic-end phase and the systolic-end phase in the temporal change specified by the temporal change acquisition unit and the calculated time ratio.

[3] The ultrasound diagnostic apparatus according to [2], in which the systolic mid-phase specifying unit specifies the systolic mid-phase by regarding a product of the elapsed time from the diastolic-end phase to the systolic-end phase in the temporal change acquisition unit and the time ratio as an elapsed time from the diastolic-end phase to the systolic mid-phase.

[4] The ultrasound diagnostic apparatus according to [3], in which the systolic mid-phase specifying unit calculates the elapsed time from the diastolic-end phase to the systolic mid-phase in a cardiac cycle immediately before a current cardiac cycle in a case in which real-time processing is performed, and specifies the systolic mid-phase of the current cardiac cycle by using the calculated elapsed time and the diastolic-end phase of the current cardiac cycle.

[5] The ultrasound diagnostic apparatus according to [1], in which the blood flow maximum phase specifying unit includes a gate setting unit that sets a Doppler gate on the ultrasound image, a blood flow calculation unit that calculates a blood flow volume by measuring a blood flow velocity based on a Doppler frequency shift at a position of the Doppler gate set by the gate setting unit, and a blood flow temporal change acquisition unit that acquires the time-varying information of the blood flow volume in the aorta based on the blood flow volume calculated by the blood flow calculation unit.

[6] The ultrasound diagnostic apparatus according to [1], in which the temporal change acquisition unit acquires a plurality of temporal changes related to a plurality of anatomical features, and the candidate frame extraction unit extracts the measurement candidate frame based on the plurality of temporal changes acquired by the temporal change acquisition unit.

[7] The ultrasound diagnostic apparatus according to [1], in which the feature recognition unit recognizes a left ventricular region of the heart as the anatomical feature, the temporal change acquisition unit acquires an area time-varying waveform of the left ventricular region as the temporal change, and the systolic mid-phase specifying unit specifies the systolic mid-phase in the area time-varying waveform by associating the blood flow maximum phase with the area time-varying waveform.

[8] The ultrasound diagnostic apparatus according to [1], in which the feature recognition unit recognizes a mitral valve annulus of the heart as the anatomical feature, the temporal change acquisition unit acquires a displacement of the mitral valve annulus as the temporal change, and the systolic mid-phase specifying unit specifies a systolic mid-phase in the displacement of the mitral valve annulus by associating the blood flow maximum phase with the displacement of the mitral valve annulus.

[9] The ultrasound diagnostic apparatus according to [1], in which the feature recognition unit recognizes an aortic valve of the heart as the anatomical feature, the temporal change acquisition unit acquires an angle change of the aortic valve as the temporal change, and the systolic mid-phase specifying unit specifies a systolic mid-phase in a displacement of a mitral valve annulus by associating the blood flow maximum phase with the angle change of the aortic valve.

[10] A control method of an ultrasound diagnostic apparatus that guides a systolic mid-phase frame from ultrasound images of a plurality of frames in which a heart of a subject is imaged, the control method including: recognizing an anatomical feature of the heart in each of the plurality of frames; acquiring a temporal change of the recognized anatomical feature; specifying a blood flow maximum phase in a cardiac cycle using time-varying information of a blood flow volume in an aorta; specifying the systolic mid-phase in the temporal change by associating the specified blood flow maximum phase with the acquired temporal change; extracting a measurement candidate frame representing the specified systolic mid-phase from the plurality of frames; and displaying the extracted measurement candidate frame in a highlighted manner on a monitor.

According to the present invention, the ultrasound diagnostic apparatus includes a feature recognition unit that recognizes an anatomical feature of the heart in each of the plurality of frames, a temporal change acquisition unit that acquires a temporal change of the anatomical feature recognized by the feature recognition unit, a blood flow maximum phase specifying unit that specifies a blood flow maximum phase in a cardiac cycle using time-varying information of a blood flow volume in an aorta, a systolic mid-phase specifying unit that specifies the systolic mid-phase in the temporal change by associating the blood flow maximum phase specified by the blood flow maximum phase specifying unit with the temporal change acquired by the temporal change acquisition unit, a candidate frame extraction unit that extracts a measurement candidate frame representing the systolic mid-phase specified by the systolic mid-phase specifying unit from the plurality of frames, a monitor, and a display controller that displays the measurement candidate frame extracted by the candidate frame extraction unit in a highlighted manner on the monitor. With this configuration, the ultrasound image of the frame representing the systolic mid-phase can be easily and accurately selected.

### BRIEF DESCRIPTION OF THE DRAWINGS

FIG. 1 is a block diagram showing a configuration of an ultrasound diagnostic apparatus according to an embodiment of the present invention.

FIG. 2 is a block diagram showing an internal configuration of a transmission and reception circuit in an embodiment of the present invention.

FIG. 3 is a block diagram showing an internal configuration of an image generation unit in an embodiment of the present invention.

FIG. 4 is a diagram showing an example of an ultrasound image representing a parasternal left ventricular long-axis cross section.

FIG. 5 is a diagram showing an example of an ultrasound image representing an apical five-chamber cross section.

FIG. 6 is a graph showing an example of an area time-varying waveform of a left ventricular region in the ultrasound image representing the parasternal left ventricular long-axis cross section.

FIG. 7 is a block diagram showing an internal configuration of a blood flow maximum phase specifying unit.

FIG. 8 is a diagram showing an example of a Doppler gate set on the ultrasound image representing the apical five-chamber cross section.

FIG. 9 is a graph showing an example of time-varying information of a blood flow volume in an aorta in a left ventricular outflow tract included in the ultrasound image representing the apical five-chamber cross section.

FIG. 10 is a diagram showing an example of a systolic mid-phase specified in the area time-varying waveform of the left ventricular region.

FIG. 11 is a diagram showing an example of an ultrasound image of a measurement candidate frame in a highlighted manner.

FIG. 12 is a flowchart showing an operation of the ultrasound diagnostic apparatus according to Embodiment of the present invention.

### DESCRIPTION OF THE PREFERRED EMBODIMENTS

Hereinafter, embodiments of the present invention will be described based on the accompanying drawings.

The following configuration requirements are described based on a representative embodiment of the present invention, but the present invention is not limited to the embodiment.

In the present specification, a numerical range represented by “to” means a range including numerical values described before and after “to”, both ends inclusive, as a lower limit value and an upper limit value.

In the present specification, “the same” includes an error range generally allowed in the technical field.

### Embodiment

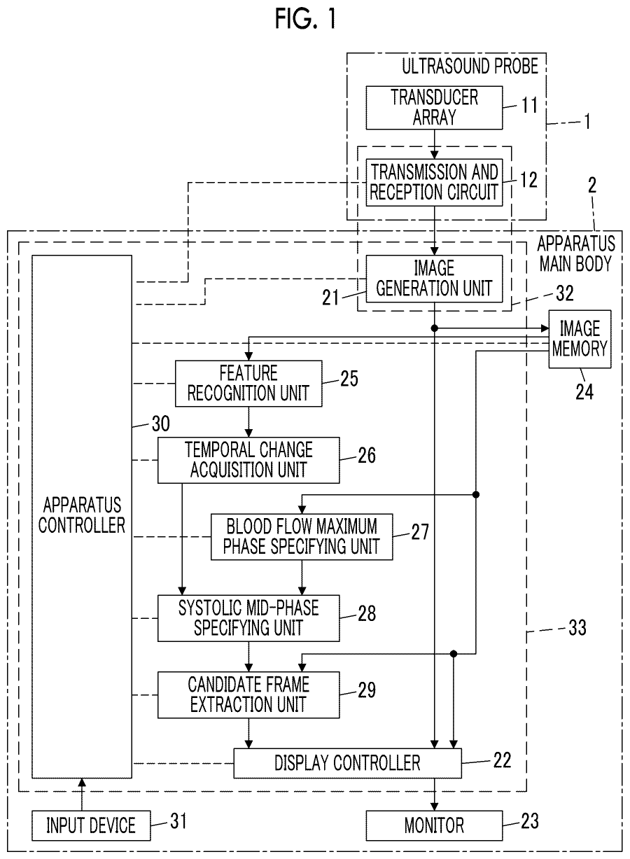

FIG. 1 shows a configuration of an ultrasound diagnostic apparatus according to an embodiment of the present invention. The ultrasound diagnostic apparatus comprises an ultrasound probe 1 and an apparatus body 2 that are connected to each other by so-called wired communication or so-called wireless communication.

The ultrasound probe 1 comprises a transducer array 11 and a transmission and reception circuit 12 connected to the transducer array 11.

The apparatus main body 2 comprises an image generation unit 21 connected to the transmission and reception circuit 12. In the apparatus main body 2, a display controller 22 and a monitor 23 are sequentially connected to the image generation unit 21. An image memory 24 is connected to the image generation unit 21. A feature recognition unit 25 is connected to the image memory 24. A temporal change acquisition unit 26 is connected to the feature recognition unit 25. In addition, a blood flow maximum phase specifying unit 27 is connected to the image memory 24. A systolic mid-phase specifying unit 28 is connected to the temporal change acquisition unit 26 and the blood flow maximum phase specifying unit 27. A candidate frame extraction unit 29 is connected to the image memory 24 and the systolic mid-phase specifying unit 28. The candidate frame extraction unit 29 is connected to the display controller 22. In addition, the image memory 24 is connected to the display controller 22. In addition, an apparatus controller 30 is connected to the transmission and reception circuit 12, the image generation unit 21, the display controller 22, the image memory 24, the feature recognition unit 25, the temporal change acquisition unit 26, the blood flow maximum phase specifying unit 27, the systolic mid-phase specifying unit 28, and the candidate frame extraction unit 29. An input device 31 is connected to the apparatus controller 30.

The transmission and reception circuit 12 and the image generation unit 21 constitute an image acquisition unit 32. In addition, the image generation unit 21, the display controller 22, the feature recognition unit 25, the temporal change acquisition unit 26, the blood flow maximum phase specifying unit 27, the systolic mid-phase specifying unit 28, the candidate frame extraction unit 29, and the apparatus controller 30 constitute a processor 33 for the apparatus main body 2.

The transducer array 11 of the ultrasound probe 1 has a plurality of ultrasonic transducers arranged in a one-dimensional or two-dimensional manner. In accordance with a drive signal supplied from the transmission and reception circuit 12, each of the ultrasound transducers transmits ultrasound and receives an ultrasound echo from a subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 32, which is composed of the transmission and reception circuit 12 and the image generation unit 21, acquires ultrasound images of a plurality of frames as a moving image in which a heart of the subject is imaged, by transmitting and receiving ultrasound beams using the ultrasound probe 1.

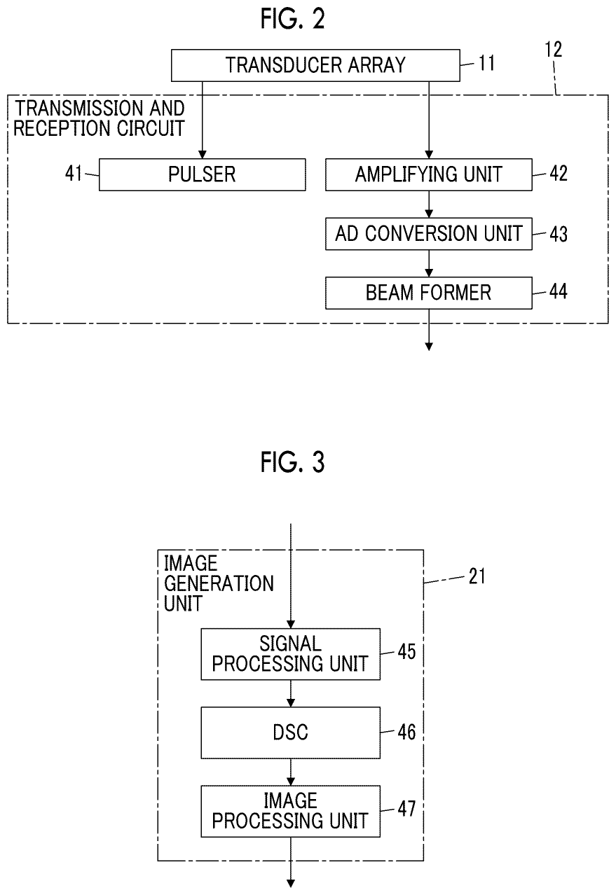

The transmission and reception circuit 12 transmits the ultrasound waves from the transducer array 11 and generates a sound ray signal based on reception signals acquired by the transducer array 11 under control of the apparatus controller 30. As shown in FIG. 2, the transmission and reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus controller 30 so that the ultrasound waves transmitted from the plurality of ultrasound oscillators of the transducer array 11 form an ultrasound beam, and supplies each drive signal to the plurality of ultrasound oscillators. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplifying unit 42.

The amplifying unit 42 amplifies the signal input from each of the ultrasound transducers constituting the transducer array 11 and transmits the amplified signal to the AD conversion unit 43. The AD conversion unit 43 converts the signal transmitted from the amplifying unit 42 into digital reception data. The beam former 44 performs so-called reception focus processing by applying and adding the delay to each reception data received from the AD conversion unit 43. By the reception focus processing, each reception data, which is converted by the AD conversion unit 43, is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation by distance of the sound ray signal received from the transmission and reception circuit 12 in accordance with depths of reflection positions of the ultrasound waves using a sound speed value set by the apparatus controller 30 and then performs envelope detection processing on the sound ray signal to generate a B-mode image signal that is tomographic image information related to tissues inside the subject.

The DSC 46 converts (raster-converts) the B-mode image signal, which is generated by the signal processing unit 45, into the image signal in accordance with a normal television signal scanning method.

The image processing unit 47 performs various types of necessary image processing such as gradation processing on the B-mode image signal input from the DSC 46, and then transmits the B-mode image signal to the display controller 22 and the image memory 24. Hereinafter, the B-mode image signal, which is image-processed by the image processing unit 47, will be referred to as an ultrasound image.

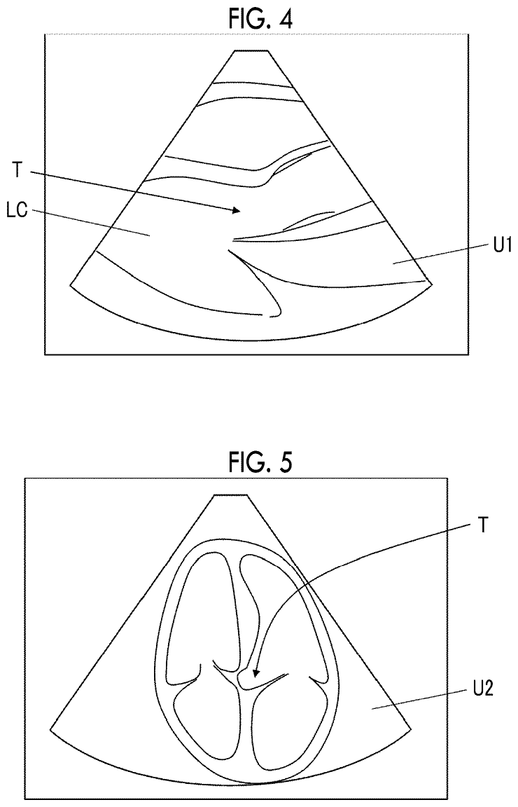

In the present invention, the image acquisition unit 32 acquires a first ultrasound image U1 representing a so-called parasternal left ventricular long-axis cross section as shown in FIG. 4 and a second ultrasound image U2 representing a so-called apical five-chamber cross section as shown in FIG. 5. The first ultrasound image U1 includes a left ventricular region LC and a left ventricular outflow tract T. The second ultrasound image U2 includes five lumens of the left ventricle, the left ventricular outflow tract T, the left atrium, the right ventricle, and the right atrium.

A technique of calculating a so-called cardiac output by capturing an ultrasound image representing a tomographic plane of a heart of a subject using an ultrasound diagnostic apparatus and analyzing the captured ultrasound image is known. The cardiac output is usually calculated by performing calculation steps of (1) measuring a diameter of a left ventricular outflow tract T in a first ultrasound image U1 of a frame representing a so-called parasternal left ventricular long-axis cross section of a systolic mid-phase of the heart, and calculating a cross-sectional area of the left ventricular outflow tract T, (2) calculating a velocity-time integral value of blood flow in the left ventricular outflow tract T for a so-called apical five-chamber cross section or a so-called apical three-chamber cross section by a so-called pulse Doppler method, (3) calculating a so-called stroke volume as a product of the cross-sectional area of the left ventricular outflow tract T and the velocity-time integral value of the blood flow in the left ventricular outflow tract T, and (4) calculating the cardiac output as a product of the stroke volume and a heart rate.

In a case in which the stroke volume or cardiac output is calculated in this way, for example, a problem may occur in which a criterion for selecting the ultrasound image of the frame representing the systolic mid-phase of the heart varies for each user of the ultrasound diagnostic apparatus, such as a doctor, or a large amount of time is required to select the ultrasound image of the frame representing the systolic mid-phase of the heart.

Here, generally, a phase in which the blood flow volume in the aorta is maximal may be regarded as the systolic mid-phase of the heart. Therefore, as will be described below, in the present invention, the first ultrasound image U1 corresponding to the systolic mid-phase is specified by associating a time-varying waveform of an area of the left ventricular region LC calculated based on the first ultrasound image U1 representing the parasternal left ventricular long-axis cross section with time-varying information of the blood flow volume in the aorta calculated based on the second ultrasound image U2 representing the apical five-chamber cross section.

The image memory 24 is a memory that stores the first ultrasound image U1 of the plurality of frames representing the parasternal left ventricular long-axis cross section acquired by the image acquisition unit 32 and the second ultrasound image U2 of the plurality of frames representing the apical five-chamber cross section acquired by the image acquisition unit 32. For example, a recording medium, such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory), can be used as the image memory 24.

The feature recognition unit 25 recognizes an anatomical feature of the heart in the first ultrasound image U1 of the plurality of frames. The feature recognition unit 25 can recognize, for example, the left ventricular region LC of the heart as the anatomical feature of the heart. The feature recognition unit 25 can perform image recognition of the left ventricular region LC of the heart by, for example, a so-called template matching method of searching for the left ventricular region LC of the heart in the first ultrasound image U1 using template data representing a typical image pattern of the left ventricular region LC of the heart, which is stored in advance. The feature recognition unit 25 can also input the first ultrasound image U1 to a trained model in so-called machine learning using a large number of ultrasound images representing a left ventricular region LC of the heart as training data, and can cause the trained model to output, to the feature recognition unit 25, an image portion representing the left ventricular region LC in the first ultrasound image U1.

The feature recognition unit 25 can also recognize, for example, a so-called mitral valve annulus or a so-called aortic valve as the anatomical feature of the heart. The feature recognition unit 25 can recognize the mitral valve annulus or the aortic valve by the same method as the recognition of the left ventricular region LC of the heart, such as a template matching method or a method using a trained model in machine learning.

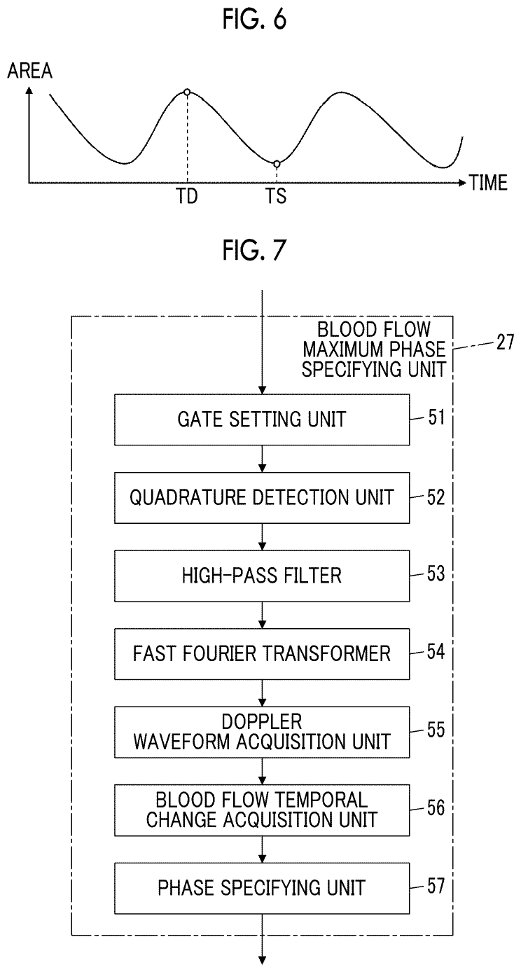

The temporal change acquisition unit 26 acquires a temporal change of the anatomical feature of the heart recognized by the feature recognition unit 25 for the first ultrasound image U1 of the plurality of frames that is continuously acquired by the image acquisition unit 32. For example, in a case in which the left ventricular region LC of the heart is recognized by the feature recognition unit, the temporal change acquisition unit 26 calculates the area of the left ventricular region LC, and arranges a plurality of values of the area of the left ventricular region LC in time series corresponding to the first ultrasound images U1 of the plurality of frames to generate an area time-varying waveform of the left ventricular region LC as shown in FIG. 6 as the temporal change of the anatomical feature.

Since the left ventricular region LC contracts and expands periodically due to the pulsation of the heart, the area time-varying waveform periodically changes to repeat a maximal value and a minimal value. The maximal value of the area time-varying waveform corresponds to a diastolic-end phase TD of the heart, and the minimal value corresponds to an systolic-end phase TS of the heart. The temporal change acquisition unit 26 specifies a phase of the maximal value in the area time-varying waveform of the left ventricular region LC as the diastolic-end phase TD, and specifies a phase of the minimal value in the area time-varying waveform as the systolic-end phase TS.

In addition, the mitral valve annulus is usually displaced in accordance with the pulsation of the heart. In the first ultrasound image U1 representing the parasternal left ventricular long-axis cross section, the mitral valve annulus is usually displaced left and right along a horizontal direction of the image orthogonal to a depth direction, and is located at the rightmost position at the diastolic-end phase and at the leftmost position at the systolic-end phase. Therefore, in a case in which the mitral valve annulus is recognized by the feature recognition unit 25, the temporal change acquisition unit 26 acquires the displacement of the mitral valve annulus in the cardiac cycle as the temporal change of the anatomical feature. The temporal change acquisition unit 26 specifies a phase in which the mitral valve annulus is located at the rightmost position in the first ultrasound image U1 representing the parasternal left ventricular long-axis cross section as the diastolic-end phase TD, and specifies a phase in which the mitral valve annulus is located at the leftmost position as the systolic-end phase TS.

In addition, the mitral valve is in an open state and the aortic valve is in a closed state in the diastole of the heart, and the mitral valve is in a closed state and the aortic valve is in an open state in the systole of the heart. Therefore, the mitral valve is closed and the aortic valve is opened at the diastole-end of the heart, and the mitral valve is opened and the aortic valve is closed at the systole-end of the heart. Therefore, in a case in which the mitral valve or the aortic valve is recognized by the feature recognition unit 25, the temporal change acquisition unit 26 acquires the angle change of the mitral valve or the angle change of the aortic valve in the cardiac cycle as the temporal change of the anatomical feature. The temporal change acquisition unit 26 specifies a phase in which the mitral valve is closed or a phase in which the aortic valve is opened as the diastolic-end phase TD, and specifies a phase in which the mitral valve is opened or a phase in which the aortic valve is closed as the systolic-end phase TS.

The blood flow maximum phase specifying unit 27 measures the blood flow volume in the aorta for at least one cardiac cycle or longer by a so-called pulse Doppler method using the second ultrasound image U2 representing the apical five-chamber cross section, generates time-varying information of the measured blood flow volume in the aorta, and specifies a blood flow maximum phase in the cardiac cycle, that is, a phase in which the blood flow volume in the aorta is maximal in one cardiac cycle, by using the generated time-varying information of the blood flow volume in the aorta. As shown in FIG. 7, the blood flow maximum phase specifying unit 27 has a configuration in which a gate setting unit 51, a quadrature detection unit 52, a high-pass filter 53, a fast Fourier transformer 54, a Doppler waveform acquisition unit 55, a blood flow temporal change acquisition unit 56, and a phase specifying unit 57 are sequentially connected.

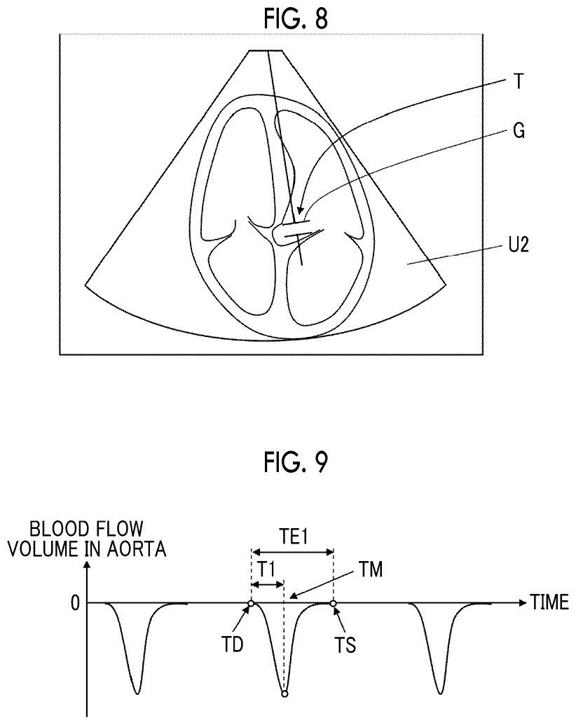

As shown in FIG. 8, the gate setting unit 51 sets a so-called Doppler gate G used to calculate the velocity-time integral value of the blood flow by the pulse Doppler method on the second ultrasound image U2 acquired by the image acquisition unit 32.

The gate setting unit 51 can set the Doppler gate G at a position designated by the user via the input device 31, for example. In addition, the gate setting unit 51 can also recognize the left ventricular outflow tract T shown in the second ultrasound image U2 by analyzing the second ultrasound image U2, and set the Doppler gate G to the recognized left ventricular outflow tract T, for example. In this case, the gate setting unit 51 can recognize the left ventricular outflow tract T by, for example, a so-called template matching method of searching the second ultrasound image U2 using a typical image pattern of the left ventricular outflow tract T in the apical five-chamber cross section, which is stored, or a method using a so-called trained model in machine learning trained on an image pattern of the left ventricular outflow tract T in the apical five-chamber cross section.

The quadrature detection unit 52 performs quadrature detection on the sound ray signal by mixing the sound ray signal received from the transmission and reception circuit 12 with a carrier signal of a reference frequency, and converts the sound ray signal into a complex signal.

The high-pass filter 53 functions as a so-called wall filter, and removes a frequency component derived from the motion of the body tissue inside the subject, from the complex signal generated by the quadrature detection unit 52.

The fast Fourier transformer 54 performs a Fourier transform on the complex data of a plurality of sample points to perform frequency analysis, obtains the blood flow velocity, and generates a spectrum signal.

The Doppler waveform acquisition unit 55 acquires a Doppler waveform image signal by representing the magnitude of each frequency component with brightness while aligning the spectrum signal generated by the fast Fourier transformer 54 on a time axis. The Doppler waveform image signal is called a so-called Doppler image, in which a time axis is shown on a horizontal axis, a Doppler shift frequency, that is, a flow velocity is shown on a vertical axis, and the brightness of the waveform represents power at each frequency component.

The blood flow temporal change acquisition unit 56 calculates the blood flow volume in the aorta in each phase by measuring the diameter of the left ventricular outflow tract T shown in the second ultrasound image U2 of the plurality of frames, and calculating a product of a value of the diameter of the left ventricular outflow tract T and a value of the flow velocity in the Doppler waveform image signal, which correspond to the same second ultrasound image U2. The blood flow temporal change acquisition unit 56 acquires time-varying information of the blood flow volume in the aorta as shown in FIG. 9 by arranging the calculated blood flow volume in the aorta in each phase in time series. In the graph representing the time-varying information of the blood flow volume in the aorta, a phase in which the value of the blood flow volume in the aorta starts to change from zero to a negative value representing the forward flow corresponds to the diastolic-end phase TD, a phase in which the blood flow volume in the aorta is minimal corresponds to the systolic mid-phase TM, and a phase in which the value of the blood flow volume in the aorta returns to zero corresponds to the systolic-end phase TS.

The blood flow temporal change acquisition unit 56 can recognize the left ventricular outflow tract T shown in the second ultrasound image U2 by, for example, a template matching method or a method using a trained model in machine learning for the second ultrasound image U2 acquired by the image acquisition unit 32, and calculate the diameter thereof.

The phase specifying unit 57 specifies a phase in which the blood flow volume in the aorta is minimal in the time-varying information of the blood flow volume in the aorta acquired by the blood flow temporal change acquisition unit 56 as the blood flow maximum phase.

The processing of specifying the blood flow maximum phase in the cardiac cycle by the gate setting unit 51, the quadrature detection unit 52, the high-pass filter 53, the fast Fourier transformer 54, the Doppler waveform acquisition unit 55, the blood flow temporal change acquisition unit 56, and the phase specifying unit 57 is performed after the drive signal is transmitted to the transducer array 11 to transmit the ultrasound waves in a pulse shape under the control of the transmission and reception circuit 12 and the apparatus controller 30.

The systolic mid-phase specifying unit 28 specifies the systolic mid-phase TM in the temporal change of the anatomical feature of the heart by associating the blood flow maximum phase specified by the blood flow maximum phase specifying unit 27 with the temporal change of the anatomical feature of the heart acquired by the temporal change acquisition unit 26.

The systolic mid-phase specifying unit 28 specifies, for example, a phase in which the forward flow starts increasing from zero and a phase in which the forward flow ends decreasing to zero as the diastolic-end phase TD and the systolic-end phase TS, respectively, based on the time-varying information of the blood flow volume in the aorta, and calculates a time ratio (T1/TE1) of an elapsed time T1 from the diastolic-end phase TD to the blood flow maximum phase specified by the blood flow maximum phase specifying unit 27 to an elapsed time TE1 from the diastolic-end phase TD to the systolic-end phase TS. The systolic mid-phase specifying unit 28 can further specify the systolic mid-phase TM based on the diastolic-end phase TD and the systolic-end phase TS in the temporal change of the anatomical feature of the heart specified by the temporal change acquisition unit 26 and the calculated time ratio (T1/TE1).

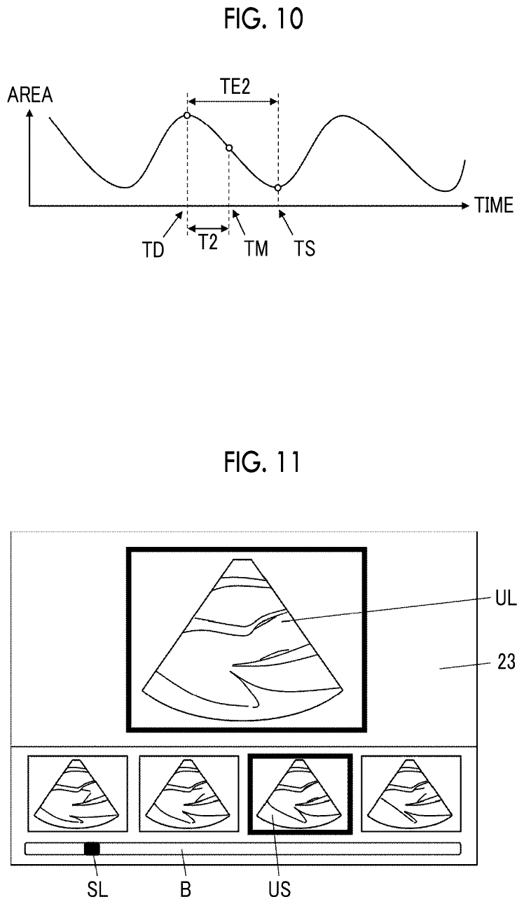

In a case in which the area time-varying waveform of the left ventricular region LC is acquired as the temporal change of the anatomical feature of the heart, the systolic mid-phase specifying unit 28 can specify the systolic mid-phase TM by regarding, for example, as shown in FIG. 10, a product of an elapsed time TE2 from the diastolic-end phase TD to the systolic-end phase TS in the area time-varying waveform of the left ventricular region LC and the time ratio (T1/TE1) as an elapsed time T2 from the diastolic-end phase TD to the systolic mid-phase TM. That is, the systolic mid-phase specifying unit 28 can calculate a phase obtained by adding the elapsed time T2 to the diastolic-end phase TD in the area time-varying waveform of the left ventricular region LC as the systolic mid-phase TM by setting (elapsed time T2)=(elapsed time TE2)×[time ratio (T1/TE1)]. Even in a case in which the displacement of the mitral valve annulus, the angle change of the aortic valve, and the like are acquired as the temporal change of the anatomical feature of the heart, the systolic mid-phase specifying unit 28 can specify the systolic mid-phase TM in the same manner as in a case in which the area time-varying waveform of the left ventricular region LC is acquired.

The candidate frame extraction unit 29 extracts the measurement candidate frame representing the systolic mid-phase TM specified by the systolic mid-phase specifying unit 28 from the first ultrasound image U1 of the plurality of frames acquired by the image acquisition unit 32.

The display controller 22 performs predetermined processing on the first ultrasound image U1, the second ultrasound image U2, and the like acquired by the image acquisition unit 32, to display the first ultrasound image U1, the second ultrasound image U2, and the like on the monitor 23 under the control of the apparatus controller 30. In addition, the display controller 22 displays the measurement candidate frame extracted by the candidate frame extraction unit 29 in a highlighted manner on the monitor 23. For example, as shown in FIG. 11, in so-called cine playback in which the first ultrasound image U1 of the plurality of frames that is already acquired is sequentially played back as a video, the display controller 22 can display the first ultrasound image U1 of the measurement candidate frame in the first ultrasound image U1 of the plurality of frames in a highlighted manner.

FIG. 11 shows an example in which a first ultrasound image UL that is sequentially displayed in time series as a video, first ultrasound images US of a plurality of frames that are sequentially displayed in time series in a scroll manner, a scroll bar B having a thin and elongated shape extending along an extension direction and each position in the extension direction corresponding to each time point from a start time point of acquisition to an end time point of acquisition of the ultrasound images U of the plurality of frames, and a slider SL that moves along the extension direction on the scroll bar B in a case in which the first ultrasound image UL that is sequentially displayed in time series as a video is acquired are displayed on the monitor 23.

The display controller 22 can display the first ultrasound image US of the measurement candidate frame in the first ultrasound image US of the plurality of frames that are sequentially displayed in the scroll display in a highlighted manner. In addition, the display controller 22 can also display the first ultrasound image US corresponding to the frame in a highlighted manner at a timing at which the first ultrasound image UL of the measurement candidate frame is displayed as the first ultrasound image UL that is sequentially displayed as a video. In this case, the first ultrasound image UL corresponding to the first ultrasound image US of the measurement candidate frame is also displayed in a highlighted manner. In the example of FIG. 11, the first ultrasound image US of four frames representing the anatomical structure of the heart that gradually changes in time series is shown.

The display controller 22 can display the first ultrasound image US of the measurement candidate frame in a highlighted manner by making a display aspect of a frame line of the first ultrasound image US of the measurement candidate frame different from a display aspect of a frame line of the first ultrasound image US of the other frames, making a display color and a brightness of the first ultrasound image US of the measurement candidate frame different from a display color and a brightness of the first ultrasound image US of the other frames, or the like.

The display aspect of the frame line of the first ultrasound image US includes a form of the frame line, such as a solid line or a dotted line, and a display color and a brightness of the frame line. In addition, the display controller 22 can also display the first ultrasound image US of the measurement candidate frame in a highlighted manner by displaying, for example, a message such as “This is a measurement candidate frame” in a so-called pop-up display or the like only in the vicinity of the first ultrasound image US of the measurement candidate frame.

The user can easily and accurately select the first ultrasound image US of the measurement frame representing the systolic mid-phase TM from among the first ultrasound image US of the plurality of frames including the first ultrasound image US of the measurement candidate frame by moving the slider SL along the extension direction of the scroll bar B via the input device 31 to check the first ultrasound images US of the plurality of frames including the first ultrasound image US of the measurement candidate frame displayed in a highlighted manner, via the input device 31.

The monitor 23 displays the first ultrasound image U1, the second ultrasound image U2, and the like under the control of the display controller 22, and includes, for example, a display device such as a liquid crystal display (LCD) or an organic electroluminescence display (organic EL display).

The input device 31 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing in the processor 33 is executed by any computer. In addition, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the order described above and may be changed as appropriate. Any computer may be a general-purpose computer, a computer for a specific use, a workstation, or another system capable of executing each process.

The processor 33 may be configured by one or a plurality of hardware, and the type of hardware is not limited. For example, the processor 33 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). In addition, the types of hardware may be a combination of different types of hardware. In a case where a plurality of hardware are configured to execute one or a plurality of processes of a certain processor, the plurality of hardware may be present in devices physically separated from each other, or may be present in the same device. In addition, in any of the embodiments, the order of each processing by the processor 33 is not limited to the above order, and may be changed as appropriate. The hardware is composed of an electric circuit (circuitry) in which circuit elements such as semiconductor elements are combined.

Furthermore, the program may be software such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be stored in a plurality of non-transitory computer-readable media existing in devices physically separated from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

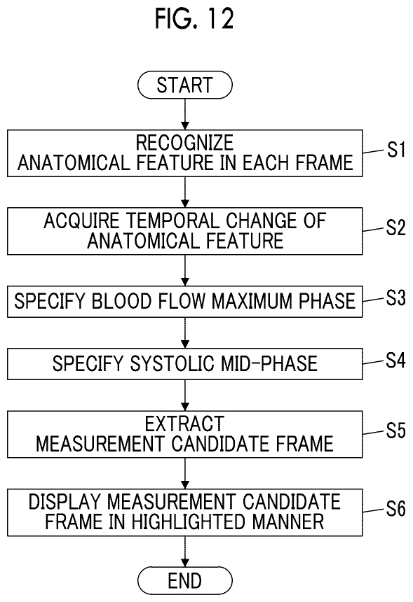

Hereinafter, an operation of the ultrasound diagnostic apparatus according to Embodiment 1 will be described with reference to a flowchart shown in FIG. 12. In the flowchart shown in FIG. 12, it is assumed that the first ultrasound image U1 of the plurality of frames representing the parasternal left ventricular long-axis cross section and the second ultrasound image U2 of the plurality of frames representing the apical five-chamber cross section have already been acquired by the image acquisition unit 32, and the first ultrasound image U1 of the plurality of frames and the second ultrasound image U2 of the plurality of frames are stored in the image memory 24. In this case, an acquisition order of the first ultrasound image U1 of the plurality of frames and the second ultrasound image U2 of the plurality of frames is not particularly limited. In addition, hereinafter, an example will be described in which the left ventricular region LC of the heart is recognized as the anatomical feature of the heart, and the area time-varying waveform of the left ventricular region LC of the heart is acquired as the temporal change of the anatomical feature.

In addition, it is assumed that the time-varying information of the blood flow volume in the aorta for at least one cardiac cycle in the apical five-chamber cross section has already been acquired by the gate setting unit 51, the quadrature detection unit 52, the high-pass filter 53, the fast Fourier transformer 54, the Doppler waveform acquisition unit 55, and the blood flow temporal change acquisition unit 56 of the blood flow maximum phase specifying unit 27.

In step S1, the feature recognition unit 25 performs image recognition of the left ventricular region LC of the heart in the first ultrasound image U1 of the plurality of frames stored in the image memory 24. The feature recognition unit 25 can perform image recognition of the left ventricular region LC of the heart by, for example, a template matching method or a method using a trained model in machine learning.

In step S2, the temporal change acquisition unit 26 calculates the area of the left ventricular region LC that is subjected to the image recognition in the first ultrasound image U1 of the plurality of frames in step S1, and arranges a plurality of values of the area of the left ventricular region LC in time series corresponding to the first ultrasound image U1 of the plurality of frames to generate the area time-varying waveform of the left ventricular region LC as shown in FIG. 6. The temporal change acquisition unit 26 further specifies a phase of the maximal value in the area time-varying waveform of the left ventricular region LC as the diastolic-end phase TD, and specifies a phase of the minimal value in the area time-varying waveform as the systolic-end phase TS.

In step S3, the blood flow maximum phase specifying unit 27 specifies the blood flow maximum phase in the cardiac cycle, that is, the phase in which the blood flow volume in the aorta is maximal in one cardiac cycle, by using the time-varying information of the blood flow volume in the aorta. In the graph representing the time-varying information of the blood flow volume in the aorta as shown in FIG. 9, a phase in which the value of the blood flow volume in the aorta starts to change from zero to a negative value representing the forward flow corresponds to the diastolic-end phase TD, a phase in which the blood flow volume in the aorta is minimal corresponds to the systolic mid-phase TM, and a phase in which the value of the blood flow volume in the aorta returns to zero corresponds to the systolic-end phase TS. Therefore, the blood flow maximum phase specifying unit 27 specifies the phase in which the blood flow volume in the aorta is minimal in the time-varying information of the blood flow volume in the aorta as the blood flow maximum phase.

In step S4, the systolic mid-phase specifying unit 28 specifies the systolic mid-phase TM in the area time-varying waveform by associating the blood flow maximum phase specified in step S3 with the area time-varying waveform generated in step S2.

In this case, the systolic mid-phase specifying unit 28 specifies, for example, a phase in which the forward flow starts increasing and a phase in which the forward flow ends decreasing as the diastolic-end phase TD and the systolic-end phase TS, respectively, based on the time-varying information of the blood flow volume in the aorta, and calculates a time ratio (T1/TE1) of an elapsed time T1 from the diastolic-end phase TD to the blood flow maximum phase specified by the blood flow maximum phase specifying unit 27 to an elapsed time TE1 from the diastolic-end phase TD to the systolic-end phase TS. The systolic mid-phase specifying unit 28 can further specify the systolic mid-phase TM based on the diastolic-end phase TD and the systolic-end phase TS in the area time-varying waveform of the left ventricular region LC specified by the temporal change acquisition unit 26 and the calculated time ratio (T1/TE1).

As shown in FIG. 10, the systolic mid-phase specifying unit 28 can specify the systolic mid-phase TM by regarding, for example, a product of an elapsed time TE2 from the diastolic-end phase TD to the systolic-end phase TS in the area time-varying waveform of the left ventricular region LC and the time ratio (T1/TE1) as an elapsed time T2 from the diastolic-end phase TD to the systolic mid-phase TM. That is, the systolic mid-phase specifying unit 28 can calculate a phase obtained by adding the elapsed time T2 to the diastolic-end phase TD in the area time-varying waveform of the left ventricular region LC as the systolic mid-phase TM by setting (elapsed time T2)=(elapsed time TE2)×[time ratio (T1/TE1)].

As described above, since the systolic mid-phase TM in the area time-varying waveform of the left ventricular region LC is specified based on the blood flow maximum phase specified in the time-varying information of the blood flow volume in the aorta, the systolic mid-phase TM in the area time-varying waveform of the left ventricular region LC can be specified under the same condition for each user and each examination, that is, the variation in the systolic mid-phase TM specified for each user and each examination can be suppressed.

In step S5, the candidate frame extraction unit 29 extracts the measurement candidate frame representing the systolic mid-phase TM specified in step S4 from the first ultrasound image U1 of the plurality of frames stored in the image memory 24.

In step S6, the display controller 22 displays the measurement candidate frame extracted in step S5 on the monitor 23 in a highlighted manner. For example, as shown in FIG. 11, in so-called cine playback in which the first ultrasound image U1 of the plurality of frames that is already acquired is sequentially played back as a video, the display controller 22 can display the first ultrasound image U1 of the measurement candidate frame in the first ultrasound image U1 of the plurality of frames in a highlighted manner.

The user can easily and accurately select the first ultrasound image US of the measurement frame representing the systolic mid-phase TM from among the first ultrasound image US of the plurality of frames including the first ultrasound image US of the measurement candidate frame by moving the slider SL along the extension direction of the scroll bar B via the input device 31 to check the first ultrasound images US of the plurality of frames including the first ultrasound image US of the measurement candidate frame displayed in a highlighted manner, via the input device 31.

In a case in which the processing of step S6 is completed in this manner, the operation of the ultrasound diagnostic apparatus according to the flowchart of FIG. 12 is completed.

As described above, according to the ultrasound diagnostic apparatus of the embodiment of the present invention, the temporal change acquisition unit 26 generates the area time-varying waveform of the left ventricular region LC subjected to the image recognition by the feature recognition unit 25, the blood flow maximum phase specifying unit 27 specifies the blood flow maximum phase in the cardiac cycle by using the time-varying information of the blood flow volume in the aorta, the systolic mid-phase specifying unit 28 specifies the systolic mid-phase TM in the area time-varying waveform by associating the blood flow maximum phase specified by the blood flow maximum phase specifying unit 27 with the area time-varying waveform generated by the temporal change acquisition unit 26, the candidate frame extraction unit 29 extracts the measurement candidate frame representing the systolic mid-phase TM specified by the systolic mid-phase specifying unit 28 from the plurality of frames, and the display controller 22 displays the measurement candidate frame extracted by the candidate frame extraction unit 29 on the monitor 23 in a highlighted manner, so that the first ultrasound image U1 of the frame representing the systolic mid-phase TM can be easily and accurately selected.

It should be noted that a case has been described in which the transmission and reception circuit 12 is provided in the ultrasound probe 1, but the transmission and reception circuit 12 may be provided in the apparatus main body 2.

In addition, although the image generation unit 21 has been described as being provided in the apparatus main body 2, the image generation unit 21 may be provided in the ultrasound probe 1.

The apparatus main body 2 may be a so-called stationary type, a portable type that is easy to carry, or a so-called handheld type that is configured by, for example, a smartphone or a tablet type computer. As described above, the type of equipment constituting the apparatus main body 2 is not particularly limited.

In the flowchart of FIG. 12, the processing of performing the image recognition of the left ventricular region LC in step S1 and the processing of generating the area time-varying waveform of the left ventricular region LC in step S2 after storing all the first ultrasound images U1 of the plurality of frames in the image memory 24 is described, but the processing of step S1 and step S2 can also be performed each time the first ultrasound image U1 is acquired.

In addition, although a case has been described in which all the second ultrasound images U2 of the plurality of frames are stored in advance in the image memory 24 and the time-varying information of the blood flow volume is acquired, for example, the blood flow maximum phase specifying unit 27 can acquire the time-varying information of the blood flow volume each time the image acquisition unit 32 acquires the second ultrasound image U2.

In addition, the first ultrasound image U1 representing the parasternal left ventricular long-axis cross section, the recognition of the anatomical feature of the heart, the acquisition of the temporal change of the anatomical feature of the heart, the specification of the systolic mid-phase TM, and the specification of the measurement candidate frame can also be performed in real time after the acquisition of the second ultrasound image U2 representing the apical five-chamber cross section, the acquisition of the time-varying information of the blood flow volume, and the specification of the blood flow maximum phase. In a case in which the real-time processing is performed, the systolic mid-phase specifying unit 28 can also calculate, for example, the elapsed time T2 from the diastolic-end phase TD to the systolic mid-phase TM in the cardiac cycle immediately before the current cardiac cycle, and specify the systolic mid-phase TM of the current cardiac cycle by using the calculated elapsed time T2 and the diastolic-end phase TD of the current cardiac cycle. As a result, the calculation load of the systolic mid-phase specifying unit 28 can be reduced.

Although a case has been described in which the temporal change acquisition unit 26 acquires the temporal change of the anatomical feature of the heart and the candidate frame extraction unit 29 extracts the measurement candidate frame based on the temporal change of the anatomical feature, the temporal change acquisition unit 26 can acquire a plurality of temporal changes related to a plurality of anatomical features, and the candidate frame extraction unit 29 can extract the measurement candidate frame based on the plurality of temporal changes generated by the temporal change acquisition unit 26.

In this case, the temporal change acquisition unit 26 can calculate, as the plurality of indices, a reduction rate of the area of the left ventricular region LC in the first ultrasound image U1 and a so-called inclination angle of the aortic valve with respect to an inner wall of the left ventricular outflow tract T in the first ultrasound image U1, in addition to the area of the left ventricular region LC in the first ultrasound image U1. The temporal change acquisition unit 26 can calculate the area of the left ventricular region LC in the first ultrasound image U1 of the plurality of frames, and calculate the reduction rate of the area of the left ventricular region LC by, for example, dividing a difference value between the areas of the left ventricular region LC in a pair of first ultrasound images U1 that are continuous in time series by a difference in acquisition time of the pair of first ultrasound images U1. In addition, the temporal change acquisition unit 26 can recognize the inner wall of the left ventricular outflow tract T and the aortic valve by, for example, a template matching method or a method using a trained model in machine learning, and calculate the angle of the aortic valve with respect to the recognized inner wall of the left ventricular outflow tract T. The temporal change acquisition unit 26 can further generate a time-varying waveform of the calculated reduction rate of the area of the left ventricular region LC, the angle of the aortic valve, and the like.

The candidate frame extraction unit 29 can extract a frame at a phase in which the reduction rate of the area of the left ventricular region LC in the first ultrasound image U1 is maximal and a frame at a phase in which the so-called inclination angle of the aortic valve with respect to the inner wall of the left ventricular outflow tract T in the first ultrasound image U1 is minimal, in addition to the frame extracted based on the area time-varying waveform of the left ventricular region LC. The candidate frame extraction unit 29 can extract the plurality of frames extracted in this way as the measurement candidate frame, and can also extract a frame at a phase corresponding to a median value or an average value of the phases of the plurality of frames as the measurement candidate frame.

Although a case has been described in which the temporal change acquisition unit 26 specifies a diastolic-end phase TD and a systolic-end phase TS in a temporal change of an anatomical feature of the heart in one cardiac cycle, the temporal change acquisition unit 26 can also specify the diastolic-end phase TD and the systolic-end phase TS in the temporal change of the anatomical feature of the heart in a plurality of cardiac cycles.

In this case, the blood flow maximum phase specifying unit 27 can specify the blood flow maximum phase in a plurality of cardiac cycles. The systolic mid-phase specifying unit 28 can specify the systolic mid-phase TM in the plurality of cardiac cycles of the temporal change of the anatomical feature of the heart based on the blood flow maximum phase specified in the plurality of cardiac cycles by the blood flow maximum phase specifying unit 27. The systolic mid-phase specifying unit 28 can also specify the systolic mid-phase TM in the temporal change of the anatomical feature of the heart by using, for example, an average value of a plurality of elapsed times T1 from the diastolic-end phase TD calculated based on the blood flow maximum phase specified in the plurality of cardiac cycles.

### EXPLANATION OF REFERENCES

1: ultrasound probe
2: apparatus main body
11: transducer array
12: transmission and reception circuit
21: image generation unit
22: display controller
23: monitor
24: image memory
25: feature recognition unit
26: temporal change acquisition unit
27: blood flow maximum phase specifying unit
28: systolic mid-phase specifying unit
29: candidate frame extraction unit
30: apparatus controller
31: input device
32: image acquisition unit
33: processor
41: pulser
42: amplifying unit
43: AD conversion unit
44: beam former
45: signal processing unit
46: DSC
47: image processing unit
51: gate setting unit
52: quadrature detection unit
53: high-pass filter
54: fast Fourier transformer
55: Doppler waveform acquisition unit
56: blood flow temporal change acquisition unit
57: phase specifying unit
B: scroll bar
G: Doppler gate
LC: left ventricular region
SL: slider
T: left ventricular outflow tract
T1, T2, TE1, TE2: elapsed time
TD: diastolic-end phase
TM: systolic mid-phase
TS: systolic-end phase
U1, UL, US: first ultrasound image
U2: second ultrasound image

What is claimed is:

1. An ultrasound diagnostic apparatus that guides a systolic mid-phase frame from ultrasound images of a plurality of frames in which a heart of a subject is imaged, the ultrasound diagnostic apparatus comprising:
a processor configured to:
recognize an anatomical feature of the heart in each of the plurality of frames;
acquire a temporal change of the anatomical feature;
specify a blood flow maximum phase in a cardiac cycle using time-varying information of a blood flow volume in an aorta;
specify the systolic mid-phase in the temporal change by associating the blood flow maximum phase with the temporal change;
extract a measurement candidate frame representing the systolic mid-phase from the plurality of frames; and
display the measurement candidate frame in a highlighted manner.

2. The ultrasound diagnostic apparatus according to claim 1,
wherein the processor is configured to:
specify a diastolic-end phase and an systolic-end phase in the temporal change;
specify a phase at which forward flow starts increasing and a phase at which the forward flow ends decreasing as the end-diastolic phase and the systolic-end phase, respectively, based on the time-varying information of the blood flow volume in the aorta;
calculate a time ratio of an elapsed time from the diastolic-end phase to the blood flow maximum phase to an elapsed time from the diastolic-end phase to the systolic-end phase;, and
specify the systolic mid-phase based on the end-diastolic phase and the systolic-end phase in the temporal change and the calculated time ratio.

3. The ultrasound diagnostic apparatus according to claim 2,
wherein the processor is configured to specify the systolic mid-phase by regarding a product of the elapsed time from the diastolic-end phase to the systolic-end phase in the temporal change and the time ratio as an elapsed time from the diastolic-end phase to the systolic mid-phase.

4. The ultrasound diagnostic apparatus according to claim 3,
wherein the processor is configured to in performing real-time processing:
calculate the elapsed time from the diastolic-end phase to the systolic mid-phase in a cardiac cycle immediately before a current cardiac cycle; and
specify the systolic mid-phase of the current cardiac cycle by using the calculated elapsed time and the diastolic-end phase of the current cardiac cycle.

5. The ultrasound diagnostic apparatus according to claim 1,
wherein the processor is configured to:
set a Doppler gate on the ultrasound image;
measure a blood flow velocity at a position of the Doppler gate; and
acquire the time-varying information of the blood flow volume in the aorta based on the measured blood flow velocity.

6. The ultrasound diagnostic apparatus according to claim 2,
wherein the processor is configured to:
set a Doppler gate on the ultrasound image;
measure a blood flow velocity at a position of the Doppler gate; and
acquire the time-varying information of the blood flow volume in the aorta based on the measured blood flow velocity.

7. The ultrasound diagnostic apparatus according to claim 3,
wherein the processor is configured to:
set a Doppler gate on the ultrasound image;
measure a blood flow velocity at a position of the Doppler gate; and
acquire the time-varying information of the blood flow volume in the aorta based on the measured blood flow velocity.

8. The ultrasound diagnostic apparatus according to claim 4,
wherein the processor is configured to:
set a Doppler gate on the ultrasound image;
measure a blood flow velocity at a position of the Doppler gate; and
acquire the time-varying information of the blood flow volume in the aorta based on the measured blood flow velocity.

9. The ultrasound diagnostic apparatus according to claim 1,
wherein the processor is configured to:
acquire a plurality of temporal changes related to a plurality of anatomical features; and
extract the measurement candidate frame based on the plurality of temporal changes.

10. The ultrasound diagnostic apparatus according to claim 2,
wherein the processor is configured to:
acquire a plurality of temporal changes related to a plurality of anatomical features; and
extract the measurement candidate frame based on the plurality of temporal changes.

11. The ultrasound diagnostic apparatus according to claim 3,
wherein the processor is configured to:
acquire a plurality of temporal changes related to a plurality of anatomical features; and
extract the measurement candidate frame based on the plurality of temporal changes.

12. The ultrasound diagnostic apparatus according to claim 4,
wherein the processor is configured to:
acquire a plurality of temporal changes related to a plurality of anatomical features; and
extract the measurement candidate frame based on the plurality of temporal changes.

13. The ultrasound diagnostic apparatus according to claim 5,
wherein the processor is configured to:
acquire a plurality of temporal changes related to a plurality of anatomical features; and
extract the measurement candidate frame based on the plurality of temporal changes.

14. The ultrasound diagnostic apparatus according to claim 1,
wherein the processor is configured to:
recognize a left ventricular region of the heart as the anatomical feature;
acquire an area time-varying waveform of the left ventricular region as the temporal change; and
specify the systolic mid-phase in the area time-varying waveform by associating the blood flow maximum phase with the area time-varying waveform.

15. The ultrasound diagnostic apparatus according to claim 2,
wherein the processor is configured to:
recognize a left ventricular region of the heart as the anatomical feature;
acquire an area time-varying waveform of the left ventricular region as the temporal change; and
specify the systolic mid-phase in the area time-varying waveform by associating the blood flow maximum phase with the area time-varying waveform.

16. The ultrasound diagnostic apparatus according to claim 1,
wherein the processor is configured to:
recognize a mitral valve annulus of the heart as the anatomical feature;
acquire a displacement of the mitral valve annulus as the temporal change; and
specify a systolic mid-phase in the displacement of the mitral valve annulus by associating the blood flow maximum phase with the displacement of the mitral valve annulus.

17. The ultrasound diagnostic apparatus according to claim 2,
wherein the processor is configured to:
recognize a mitral valve annulus of the heart as the anatomical feature;
acquire a displacement of the mitral valve annulus as the temporal change; and
specify a systolic mid-phase in the displacement of the mitral valve annulus by associating the blood flow maximum phase with the displacement of the mitral valve annulus.

18. The ultrasound diagnostic apparatus according to claim 1,
wherein the processor is configured to:
recognize an aortic valve of the heart as the anatomical feature;
acquire an angle change of the aortic valve as the temporal change; and
specify a systolic mid-phase in a displacement of a mitral valve annulus by associating the blood flow maximum phase with the angle change of the aortic valve.

19. The ultrasound diagnostic apparatus according to claim 2,
wherein the processor is configured to:
recognize an aortic valve of the heart as the anatomical feature;
acquire an angle change of the aortic valve as the temporal change; and
specify a systolic mid-phase in a displacement of a mitral valve annulus by associating the blood flow maximum phase with the angle change of the aortic valve.

20. A control method of an ultrasound diagnostic apparatus that guides a systolic mid-phase frame from ultrasound images of a plurality of frames in which a heart of a subject is imaged, the control method comprising:
recognizing an anatomical feature of the heart in each of the plurality of frames;
acquiring a temporal change of the recognized anatomical feature;
specifying a blood flow maximum phase in a cardiac cycle using time-varying information of a blood flow volume in an aorta;
specifying the systolic mid-phase in the temporal change by associating the specified blood flow maximum phase with the acquired temporal change;
extracting a measurement candidate frame representing the specified systolic mid-phase from the plurality of frames; and
displaying the extracted measurement candidate frame in a highlighted manner on a monitor.
